(19) United States
(12) Patent Application Publication
(10) Pub. No.: US 2013/0331846 A1
(43) Pub. Date: Dec. 12, 2013

(54) AUGMENTATION SYSTEM

(71) Applicants: Jay SMITH, West Chester, PA (US); Michael Cross, Alexandria, VA (US); Ray Pacheco, West Chester, PA (US); Glen Pierson, West Chester, PA (US); Sean Kerr, West Chester, PA (US)

(72) Inventors: Jay SMITH, West Chester, PA (US); Michael Cross, Alexandria, VA (US); Ray Pacheco, West Chester, PA (US); Glen Pierson, West Chester, PA (US); Sean Kerr, West Chester, PA (US)

(21) Appl. No.: 13/494,593

(22) Filed: Jun. 12, 2012

(51) Int. Cl. A61B 17/88

(52) U.S. Cl. CPC A61B 17/8822
USPC 606/93

(57) ABSTRACT

A device for bone fixation includes (a) a delivery gun having a handle and (b) an elongated cartridge coupled to the handle. The cartridge has a distal portion configured and dimensioned for insertion to a target area within a bone. The cartridge has a channel extending therethrough between a proximal end and an opening at the distal end and housing an injectable material therein. A first portion of the cartridge has a first heating element maintaining the injectable material within a first target temperature range. A second portion of the cartridge has a second heating element maintaining the injectable material within a second target temperature range.

### BACKGROUND INFORMATION

Fractures are often treated with screws or other fixation devices to stabilize the fractured portions of the bone once they have been brought into corrective alignment. Once implanted, a strengthening material may be injected into a region of the bone fixation devices to increase a holding strength thereof Conventional devices have employed injections of poly(methyl)methacrylate (“PMMA”) into the bone. However, the viscosity of this material changes rapidly as it begins to cure increasing the difficulty and reducing the efficacy these procedures. The difficulty applying this material may result in reduced anchoring strength of the bone fixation device, increasing the likelihood of further fractures or other complications.

### SUMMARY OF THE INVENTION

The present invention is directed to a device for bone fixation, comprising a cartridge defining a reservoir therein, the cartridge including a first heating element adjacent to the reservoir operable to maintain injectable material housed in the reservoir within a first target temperature range. A distal portion of the cartridge is configured and dimensioned for insertion to a target area within a bone, the cartridge having a channel extending therethrough between a proximal end and an opening at the distal end. The cartridge also includes a distal portion heating arrangement in the distal portion of the cartridge to maintain injectable material flowing therethrough within a second target temperature range.

### BRIEF DESCRIPTION OF THE DRAWINGS

FIG. 1 shows a first perspective view of an injection gun for the bone fixation system according to a first exemplary embodiment of the present invention;

FIG. 2 shows a first cross-sectional view of the injection gun of FIG. 1;

FIG. 3 shows a second perspective view of the injection gun of FIG. 1;

FIG. 4 shows a partial cross-sectional view of the coupling head and cartridge of FIG. 1;

FIG. 5 shoes a first perspective view of the cartridge of FIG. 1;

FIG. 6 shows a perspective view of the cartridge of FIG. 1 without an outer shell;

FIG. 7 shows a zoomed view of a proximal end of the cartridge of FIG. 5;

FIG. 8 shows a zoomed view of a proximal end of the cartridge of FIG. 6;

FIG. 9 shows a zoomed view of a distal end of the cartridge of FIG. 1;

FIG. 10 shows an exploded view of the cartridge of FIG. 1;

FIG. 11 shows a perspective view of a plunger assembly for use with the injection gun of FIG. 1;

FIG. 12 shows a transparent view of the plunger assembly of FIG. 11;

FIG. 13 shows a perspective view of a cartridge coupler for use with the injection gun of FIG. 1;

FIG. 14 shows a side view of a bone injection gun according to a second exemplary embodiment of the present invention;

FIG. 15 shows a cross-sectional side view of the bone injection gun of FIG. 13;

FIG. 16 shows a side view of a bone injection gun according to a third exemplary embodiment of the present invention;

FIG. 17 shows a side view of a cartridge for use with the bone injection gun of FIG. 16;

FIG. 18 shows a perspective view of a PCB according to a first alternate embodiment of the invention; and

FIG. 19 shows a perspective view of a PCB according to a second alternate embodiment of the invention.

### DETAILED DESCRIPTION

The present invention may be further understood with reference to the following description and the appended drawings. The present invention relates generally to devices and methods for the fixation and stabilization of fractures. It is noted that although embodiments of the present invention have been described with respect to particular bones, the present invention may also be employed in a variety of other bone fixation procedures including, but not limited to, maxillofacial fixation procedures and spinal fixation procedures. The present invention relates to a delivery system configured to inject a bone strengthening material through a cannula of a bone fixation device (e.g., a nail screw, a bone plate, etc.) inserted into cancellous bone tissue. Specifically, the delivery system according to the invention is configured to facilitate insertion of an anchoring material into the bone in a manner selected so that material properties (e.g., viscosity, etc.) of the inserted material are optimized for bone fixation, as will be described in greater detail later on. An exemplary delivery system according to the invention comprises a heated nozzle having heated portions disposed along a length thereof and configured to maintain a temperature of the bone strengthening material within an optimal predetermined temperature range until the bone strengthening material exits the nozzle into the bone. In an exemplary embodiment, the bone strengthening material of the present invention is polycaprolactone (“PCL”) configured to begin to cool/solidify only after exiting the nozzle of the delivery system. It is noted that although the present invention is described with respect to the fixation of a femoral bone, the exemplary delivery system and bone strengthening material may be used for the fixation of any other bone. For example, the system may be used for the treatment of a proximal humerus through a screw cannula, with a trochanteric plate implant system, through a lag screw of a distal femur, etc. It is also noted that although the exemplary embodiments specifically describe the bone strengthening material as PCL, the system of the present invention may utilize any bone strengthening material that may be melted and subsequently resolidified such as, for example, ethylene vinyl acetate (EVA) or thermoplastics. The term proximal, as used herein, refers to a direction approaching a physician or other user while the term distal refers to a direction approaching a target portion of a fractured or otherwise damaged bone.

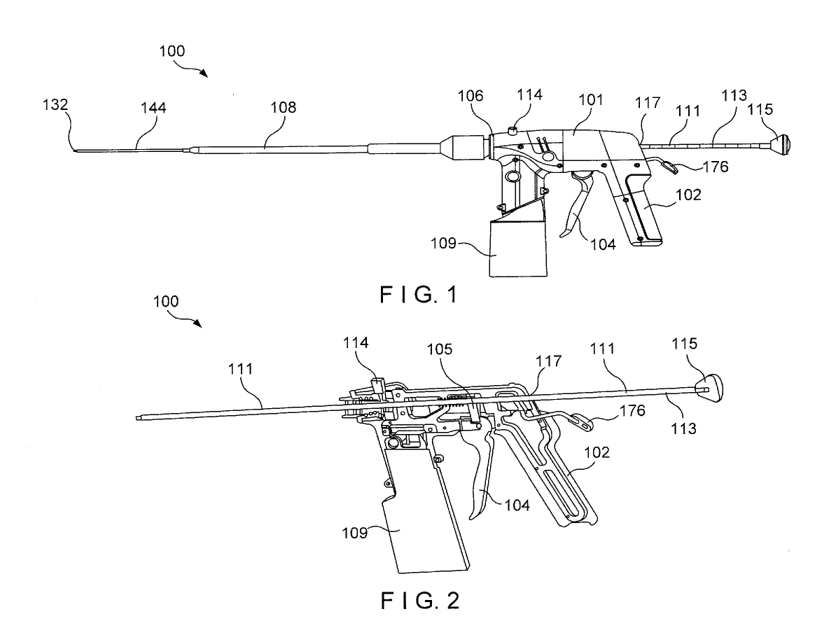

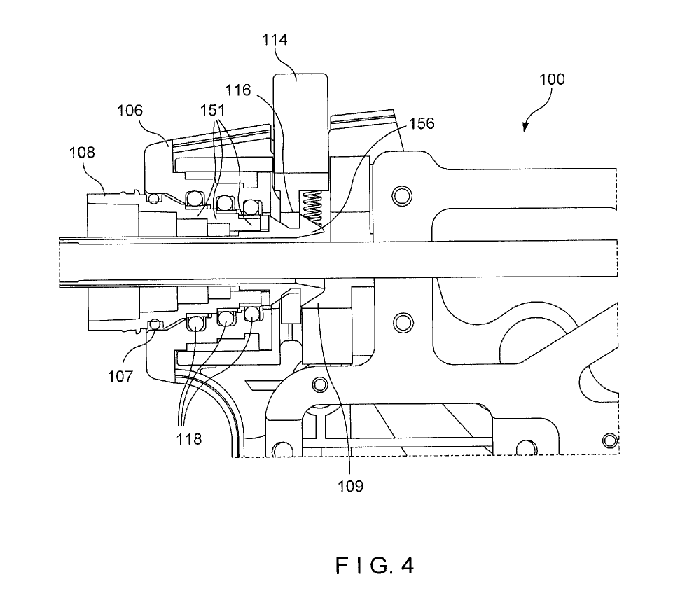

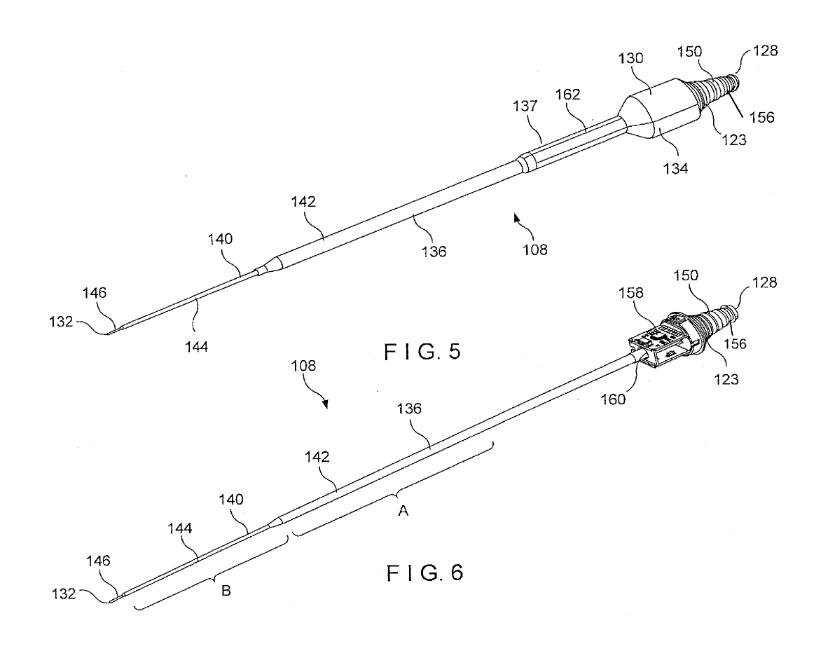

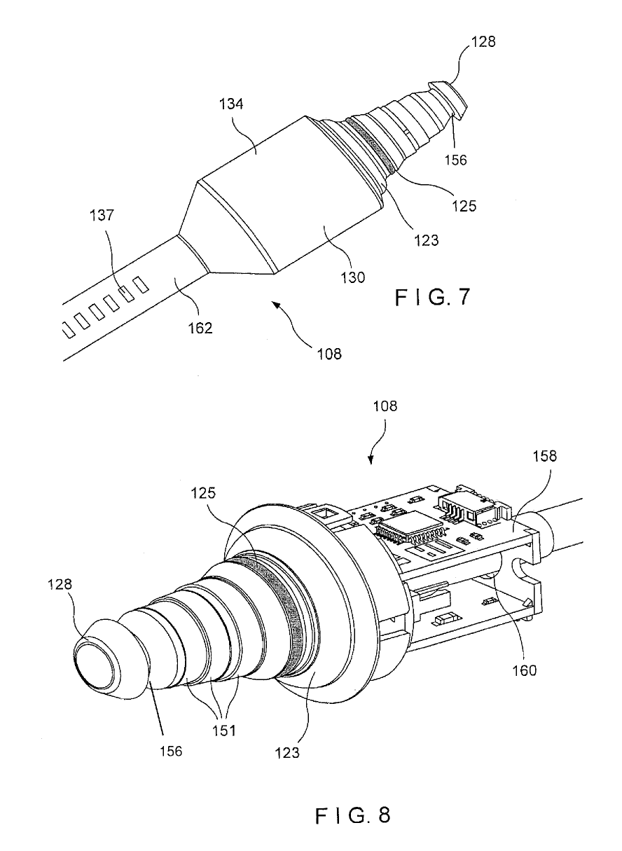

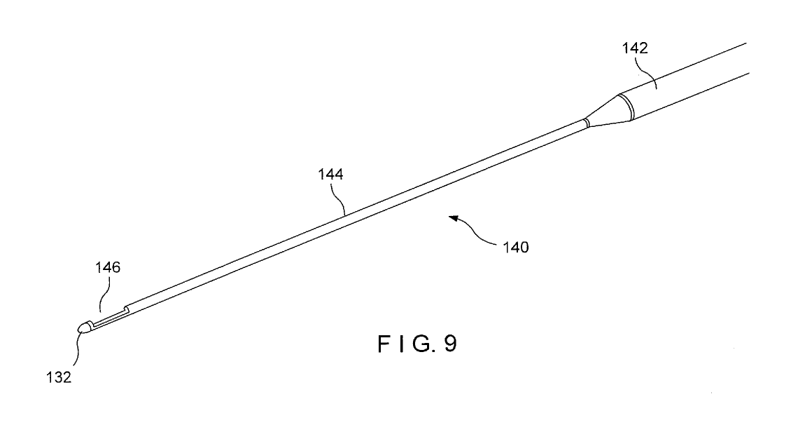

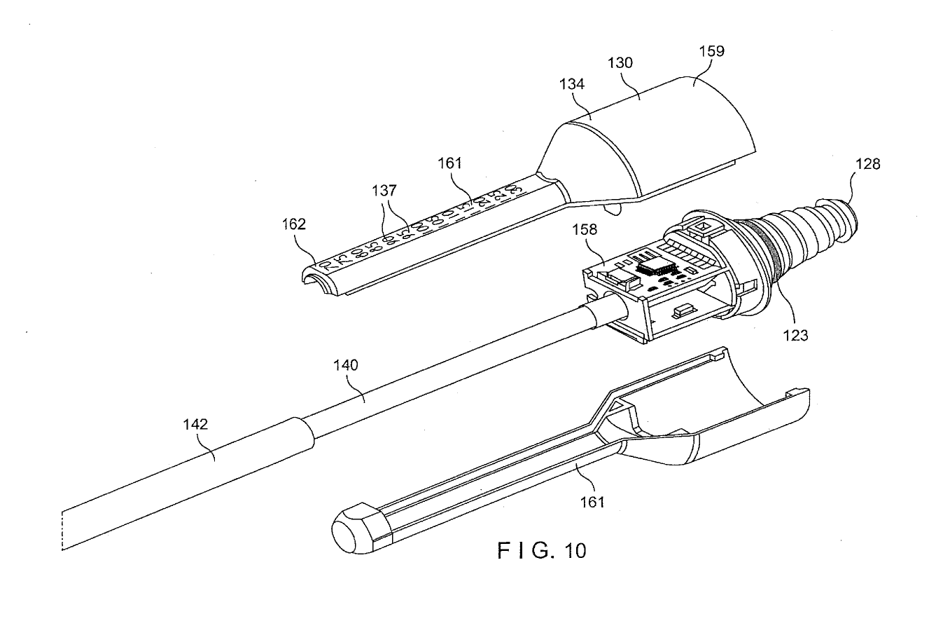

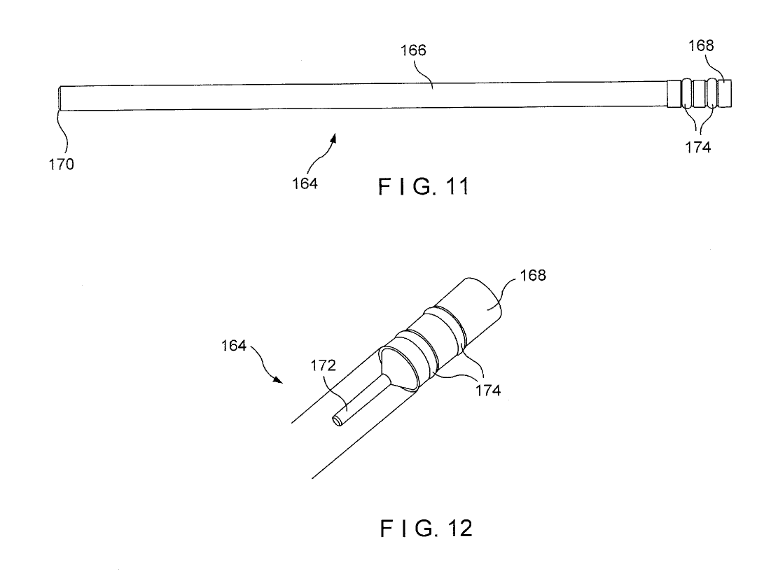

As shown in FIGS. 1-12, a delivery system 100 according to the invention comprises a body portion 101 having a handle 102 at a proximal end and including an actuator 104. A coupling portion 106 connects the handle 102 to a cartridge 108 configured for insertion into a living body to a target portion of a bone (not shown). The exemplary coupling portion 106 according to the invention comprises a substantially cylindrical frame, a proximal portion of which is integrally formed with the handle 102, as will be described in greater detail later on. The coupling portion 106 permits removable attachment of the cartridge 108 to the handle 102, thus permitting the handle 102 to be reusable in conjunction with disposable cartridges 108. That is, as only the cartridge 108 is inserted into the body, the handle 102 may be reused by coupling a sterile, disposable cartridge 108 thereto. The coupling portion 106 includes an opening 107 extending thereinto to permit insertion of the cartridge 108. Specifically, the proximal end 128 of the cartridge 108 includes a neck portion 150 configured to engage the coupling portion 106, as will be described in greater detail later on. The coupling head 106 interfaces with a proximal portion of the cartridge 108 comprising the onboard electronics, LED indicators, etc. of the system 100, as will be described in greater detail later on. Specifically, the coupling portion 106 comprises an opening 107 extending thereinto to a depth selected to detachably receive a proximal portion of the cartridge 108. The opening 107 has a stepped shape accommodating a correspondingly shaped proximal portion of the cartridge 108 and extends through the coupling portion 106 toward a proximal end of the body portion 101, terminating at a proximal end 117. As will be described in greater detail with respect to the exemplary method, the opening 107 is dimensioned to permit insertion of a push rod 164 therethrough. In an exemplary embodiment, the opening 107 is substantially cylindrical, although other cross-sectional shapes may be employed without deviating from the scope of the invention. The coupling head 106 comprises a plurality of electrical connections 118 laser welded thereto. The coupling head 106 also includes standard electric power connections (e.g., ground and live terminals) configured for connection to an external power source (not shown) or to a battery 109 mounted to the handle 102. An electronic component such as, for example, a printed circuit board 158, is also integrated into the handle 102 and connected to the electrical connections 118 via, for example, a wire (not shown). The delivery system 100 in this embodiment is coupled to a rechargeable battery pack 109 attached to handle 102. In another exemplary embodiment, the system 100 may have a built-in power source or a connection to an external source of power as will be understood by those skilled in the art. The electronic component may also be configured to power one or more LED indicators 123 on a proximal portion of the cartridge 108, so that the lighting of the LED provides information to a user on the state of the delivery system 100 (e.g., indicating that the PCL has reached a predetermined temperature, overheating of the PCL, an on/off state of the delivery system 100, etc.). For example, a yellow light may indicate that the PCL is being heated, a green light may indicate that the PCL is ready and a red light may indicate that there has been an error. Specifically, when the cartridge 108 and the coupling head 106 are engaged, the coupling head 106 is electrically connected to the cartridge 108 to provide power for the LED indicators 123. In an operative configuration, the LED indicators 123 may switch colors (e.g., red, green, amber, etc.) to indicate a current state of the device 100 and/or the cartridge 108 (e.g., battery power, a heating state of the cartridge, whether the cartridge is ready for use, etc.). The exemplary heating system according to the invention may be configured to apply a total of approximately 60 watts of heat to the PCL as it travels through the cartridge 108, the heat being supplied by a power source of approximately 14.4 v. The heating elements and LED indicators may be connected to a microcontroller (not shown) to monitor and control the amount of heat applied, as those skilled in the art will understand.

The coupling head 106 further comprises a sliding lock mechanism 114 on a side thereof to removably attach the coupling head 106 to the cartridge 108. The sliding lock mechanism 114, which is movable between an open configuration and a locking configuration, comprises a tab 116 frictionally engaging a proximal portion of the cartridge 108 inserted into the coupling head 106, as will be described in greater detail below. In an exemplary embodiment, the sliding lock mechanism is spring-loaded, as those skilled in the art will understand. The tab 116 extends into the opening 107 substantially perpendicular to a longitudinal axis of the opening 107 to apply frictional force to a proximal portion of the cartridge 108. A proximal end 128 of the cartridge 108 according to this embodiment further includes a radial groove 156 lockingly engaging the tab 116 to prevent withdrawal of the cartridge 108 from the coupling portion 106 when the sliding lock mechanism 114 has been depressed. Thus, the cartridge 108 may only be withdrawn from the coupling portion 106 by releasing the lock mechanism 114. The coupling head 106 further comprise a coaxial connector at the proximal end 111 which couples the coupling head 106 to the handle 102 so that the system 100 may be rotated while maintaining a desired orientation and depth of a distal end of the cartridge 108.

The cartridge 108 is an elongated substantially cylindrical element extending from a proximal end 128 having a neck portion 150 and a PCB connector 130 to a distal end 132. The neck 150 is configured to be received within the coupling head 106 in an operative configuration and is substantially cylindrical. A diameter of the neck 150 conforms to a diameter of the opening 107 extending into the distal face of the coupling head 106 to permit locking engagement therewith, as described in greater detail earlier. The neck 150 comprises at least one circumferential groove 156 extending into an outer body thereof to a predetermined depth. The groove 156 is positioned so that when the neck 150 is inserted into the opening 107 of the coupling head 106, the tab 116 is aligned therewith, as also described in greater detail earlier. Specifically, movement of the sliding lock 114 to a locked configuration causes the tab 116 to frictionally engage the groove 156 to lock the cartridge 108 to the coupling head 106. As those skilled in the art will understand, the circumferential groove 156 permits 360° rotation of the shaft 136 relative to the coupling head 106.

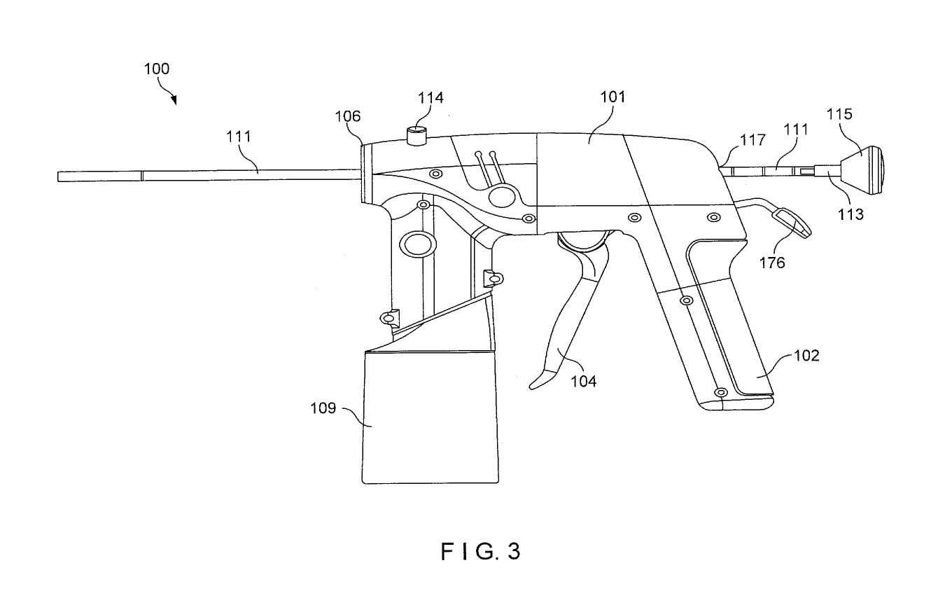

As shown in FIGS. 3-6 and 8, the PCB connector 130 extends distally from the neck 150 and has an enlarged diameter portion 134 configured to abut the distal face of the coupling head 106. An elongated shaft 136 of the cartridge 108 extends distally from the enlarged diameter portion 134 by a length sufficient to permit insertion of the distal end 132 to a desired position within a target bone to be treated. A delivery tube 140 extends through the length of the cartridge 108 and into the coupling head 106. The delivery tube 140 which houses the PCL includes a proximal portion 142 having a first diameter selected to permit insertion of a PCL rod 166 therein and to permit insertion thereof within a channel of the shaft 136. A distal portion/nozzle 144 of the delivery tube 140 extending distally of the proximal portion 142 is configured to be insertable into the target bone directly or through the cannulation of a previously placed implant. In its unheated state, the PCL rod 166 is too large to be inserted into the distal portion 144. After heating the PCL melts to a flowable state in which it may pass through the distal portion 144. In an exemplary embodiment, the proximal and distal portions 142, 144 may be formed as a single unit having a single seamless bore extending therethrough to permit uninterrupted flow of PCL therethrough. As those skilled in the art will understand, this construction has increased rigidity when compared to multi-piece elements and prevents the formation of heat sinks that might results at junctures between separately formed portions. A length of the distal portion 144 according to this embodiment is selected to prevent the distal end 132 from extending distally out of the implant. However, as would be understood by those skilled in the art, other lengths of the distal portion 144 may be selected to achieve any desired position of the distal end 132. A diameter of the distal portion 144 according to this embodiment is selected to substantially match an inner diameter of an implant through which it is to be inserted to prevent backflow of PCL into the insert, as those skilled in the art will understand.

The neck portion 150 includes a plurality of electrical connections 151 configured to engage the contacts 118 when assembled to the handle 102. As those skilled in the art will understand, this connection permits the transfer of energy through the cartridge 108 to power the heating elements, thermal controls and thermal monitors disposed throughout the cartridge 108 to achieve the desired heating of the PCL. The neck portion 150 of the cartridge 108 further comprises an O-ring 125 configured to seal the cartridge 108 to the body portion 101 in the operative configuration, as those skilled in the art will understand.

A nozzle opening 146 is formed at a distal end of the distal portion 144 to permit the PCL to exit therefrom into the bone, as will be described in greater detail later on. In an exemplary embodiment, the distal portion 144 has an internal diameter of approximately 2 mm to permit manual injection of the PCL from a distal opening thereof into the bone. It is noted that any type and size of nozzle opening 146 may be attached to the delivery tube 140 for use in different procedures. For example, the distal portion 144 and the nozzle 146 may be adapted for the screw augmentation of the proximal humerus, distal femur, proximal tibia, etc. In an alternate embodiment, the distal portion 144 extending distally of the proximal portion 142 may be flexible to permit insertion along curved or winding paths. In an exemplary embodiment, the nozzle opening 146 is oriented so that, when attached to the handle 102, the opening 146 faces a superior direction such that PCL flowing therefrom is directed to a portion of the target bone above the implant. For example, where the implant is inserted into the femoral head, the PCL mass formed above the implant increase the ability of the bone to prevent the implant from being pushed through the upper surface of the femoral head as the bone is loaded. However, as would be understood by those skilled in the art, the nozzle opening 146 may be aimed in any other orientation to direct the flow of PCL to any desired target area without deviating from the scope of the invention.

The delivery system 100 according to this embodiment may be used without being inserted through a bone implant to, for example, fill voids in a target bone. In this situation, a user may, for example, insert the distal portion 144 to a target depth within the bone and gradually retract the system 100 as the PCL is injected as will be described in greater detail later on. Cartridges 108 may be provided in various sizes so that a user may select a cartridge 108 the dimensions of which (e.g., the diameter and length of the distal portion 144) conform to the requirements of a particular procedure (e.g., the diameter and depth of a bore drilled into the target bone or the cannulation of an implant), as those skilled in the art will understand. Furthermore, the distal portion 144 may be substantially cylindrical with a uniform outer diameter or a diameter that tapers down in size in a distal direction.

The PCB connector 130 may include either a single or a multi-unit PCB 158. In one exemplary embodiment, as shown in FIG. 8, the PCB is arranged within the enlarged diameter portion 134 substantially perpendicular to a longitudinal axis of the cartridge 108. An opening 160 extending through the connector 130 permits insertion of the delivery tube 140 therethrough. The PCB 158 further comprises a debug pin connected to the microcontroller (not shown), as those skilled in the art will understand. It is noted that the PCB 158 may be arranged in any configuration within the enlarged diameter portion 134, or in any other suitable portion of the cartridge 108 without deviating from the scope of the invention. A proximal portion of the PCB 158 is configured to interface with the neck 150. The PCB may be housed within an insulated housing 159 having first and second semi-cylindrical portions 161 attached to one another.

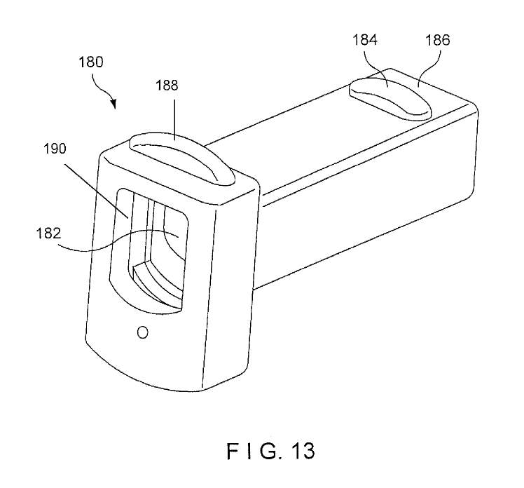

A proximal portion of the cartridge 108 distal of the PCB connector 130 may be keyed to aid in orientation of the delivery tube 140 to a desired orientation in the body. Specifically, a keyed portion 162 of the cartridge 108 may have a substantially D-shaped cross-section to prevent rotation thereof relative to an aiming arm used in the insertion of an implant (e.g., a trochanteric implant). The keyed portion 162 extends along a length of the shaft 136 such that the shaft 136 may only be inserted through an opening of the aiming arm in a desired orientation relative thereto, as will be described in greater detail later on. Thus, the keyed portion 162 ensures the desired orientation of the nozzle 146 relative to the implant in which it is inserted. The shaft 136 may also include a locking feature 137 extending about a portion of the shaft 136 at a desired position. The locking feature 137 according to this embodiment comprises a plurality of notches indicating to a user of the system 100 when the nozzle 146 has been inserted to a desired depth. The keyed portion 162 is configured to optionally receive a cartridge coupler 180 thereover, as described in greater detail with respect to FIGS. 12-13 below.

The delivery system 100 further comprises a plunger assembly 164 including a substantially cylindrical PCL rod 166 extending distally from a plunger 168. The plunger assembly 164 is configured for insertion through the delivery tube 140 in an operative configuration. The plunger 168 may be formed, for example, of stainless steel or a high temperature, high strength plastic (e.g. PEEK, Ultem, etc.) and includes one or more O-rings 174 on an outer surface thereof to provide a seal within the cartridge 108. The plunger 168 may further include a distal stem 172 extending into the PCL rod 166 to aid in assembly thereof, as shown in the transparent view of FIG. 12. The plunger assembly 164 is received within the cartridge 108 in an operative configuration. Once heated to a desired melting temperate within the cartridge 108, distal movement of the plunger 168 within the cartridge 108 moves the melted PCL of the PCL rod 166 distally out of the nozzle opening 146 of the delivery tube 140 and into the target bone. Distal movement of the plunger 168 may be controlled by a push rod 111 inserted through the opening 107 and through the body 101 into the delivery tube 140. The push rod 111 may be advanced distally into the device 100 to control the release of the PCL via the nozzle opening 146. The push rod 111 may include an elongated body portion 113 and an enlarged diameter portion 115 at a proximal end thereof. The enlarged diameter portion 115 prevents the push rod 111 from being completely advanced into the opening 107. Specifically, the actuator 104 is connected to a sliding plate 105 operably connected to the push rod 111 so that a proximally directed force on the actuator 104 is converted to a distally directed force on the push rod 111, as those skilled in the art will understand. A spring mechanism may be located on the handle 102 to bias the actuator 104 to return to an initial configuration once the proximally directed force is removed therefrom, permitting a physician or other user to advance the push rod 111 further distally as needed. The delivery system 100 may also include a release lever 176 connecting the push rod 111 to the handle 102 via an elastic element such as, for example, a spring. The release lever 176 is movable between a first locked position and a second unlocked position. In the first position, the push rod 111 is locked with respect to the handle 102 and prevented from moving distally relative thereto. In the second position, the push rod 111 is unlocked such that actuation of the actuator 104 moves the push rod 111 distally with respect to the handle 102.

In an exemplary embodiment, an entire length of the delivery tube 140 is heated, with separate portions thereof maintained at different temperatures. Specifically, the delivery tube 140 is heated so that a temperature thereof is gradually reduced toward the distal end so that the temperature of the PCL decreases from a temperature of approximately 80° C. to 85° C. at a region adjacent to the proximal portion 142 and through the shaft 136 to a temperature of approximately 60° to 65° C. at a region B of the distal portion 144 of the delivery tube 140 and at the nozzle 146. Specifically, the distal portion 144 and nozzle 146 are provided with heating elements (not shown) adjacent proximal and distal end portions thereof. The heating elements (not shown) of the distal portion 144 are configured so that a distal-most heating element applies less heat than a proximal-most heating element. For example, the delivery tube 140 may include helical windings, which may be separately controlled to heat the PCL as required.

The exemplary delivery system 100 according to the invention employs PCL to add strength and stability to a target bone. The material properties of PCL provide that resorption within the body occurs slowly over a period of approximately 2-5 years, thus permitting ample time for the bone to heal. As the elastic modulus of PCL is very similar to that of bone, the PCL provides the strength necessary to maintain stable bone fixation. PCL also has the advantage of being non-adhesive and drillable so that, once cooled within the bone, a physician or other user may drill a bore thereinto to perform other bone fixation procedures. Additionally, the use of PCL eliminates the need for materials that must undergo a chemical reaction prior to or after insertion into the bone.

In accordance with an exemplary method according to the invention, the delivery system 100 is assembled in the configuration shown in FIG. 1. A solid piece of PCL (i.e., the plunger assembly 164) is assembled in the cartridge 108 either pre-operatively or during manufacturing. The cartridge 108 is loaded onto the body portion 101 of the delivery system 100. The delivery system 100 is then moved to an on-position and the PCL rod 166 is heated to a desired optimal temperature as discussed in greater detail earlier. The LED indicator 123 indicates to the user when the PCL has been heated to the desired temperature. The LED indicator 123 indicates to the user when the PCL has been heated to the desired temperature. A bore (not shown) is drilled into a target bone to a target depth conforming to a desired positioning of a bone fixation element in the bone or a depth of a diseased or damaged portion of the bone to be treated. In another exemplary method, an implant (e.g., a trochanteric implant) with a cannula extending therethrough is inserted to a desired position within a target bone and the delivery tube 140 is inserted into the bone via the cannula in the implant. The delivery system 100 is then positioned relative to the bone so that the distal end 132 of the shaft portion 136 is seated against an outer periphery of the drilled bore (not shown). In this position, the nozzle opening 146 of the delivery tube 140 is open to the target portion of the bone. The actuator 104 is then moved proximally to move the push rod 111 and the plunger assembly 164 distally. The distal movement of the plunger assembly 164 drives the PCL distally out of the nozzle opening 146 into the target bone. The temperature of the PCL gradually decreases until the PCL cools and assumes a substantially rigid state within the bone. When used in combination with a bone implant, it is rioted that the exemplary PCL injection method disclosed above may be performed prior to or after the insertion of the bone implant into the bone. Furthermore, the bone implant may include a relief cut aligning with the nozzle opening 146 to permit the PCL to flow therefrom. The bone implant may be non-rotatably attached to the bone to aid in such alignment.

In an exemplary embodiment, the entire cartridge 108 is disposable. After performing a first PCL injection procedure, a user may optionally attach a second cartridge 108 to the body portion 101 to inject additional PCL into the same target area, into a different portion of the same bone or into an entirely different bone. Any number of cartridges may be used to conform to the requirements of a particular procedure.

In another embodiment of the invention, as shown in FIG. 13, the system 100 may be used in combination with the cartridge coupler 180. The cartridge coupler 180 is formed as an elongated element having an opening 182 extending therethrough to receive the cartridge 108. The opening 182 has a substantially D-shaped cross-section sized to engage a corresponding shape of the keyed portion 162 of the cartridge 108, as described in greater detail earlier. The cartridge coupler 180 may include a first pushbutton 184 having a tab (not shown) extending into the opening 182 by a distance selected to engage the notches 137 of the cartridge 108. A window 186 may be provided adjacent the first pushbutton 184 to permit viewing of the cartridge 108 therethrough and aid in insertion thereof into the body by a target depth. The cartridge coupler 180 may further comprise a second pushbutton 188 having a tab (not shown) extending into the opening 182 to lockingly engage an aiming arm (not shown). In an operative configuration, actuation of the second pushbutton 188 permits removal of the cartridge 108 therefrom to, for example, permit insertion of a new cartridge 108.

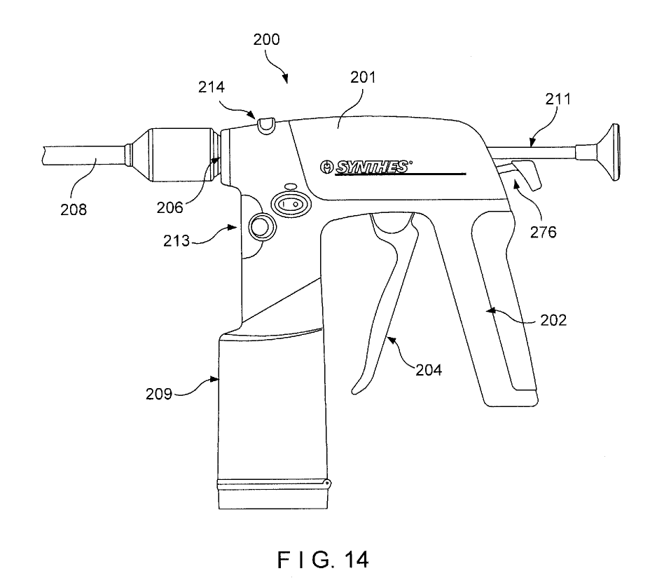

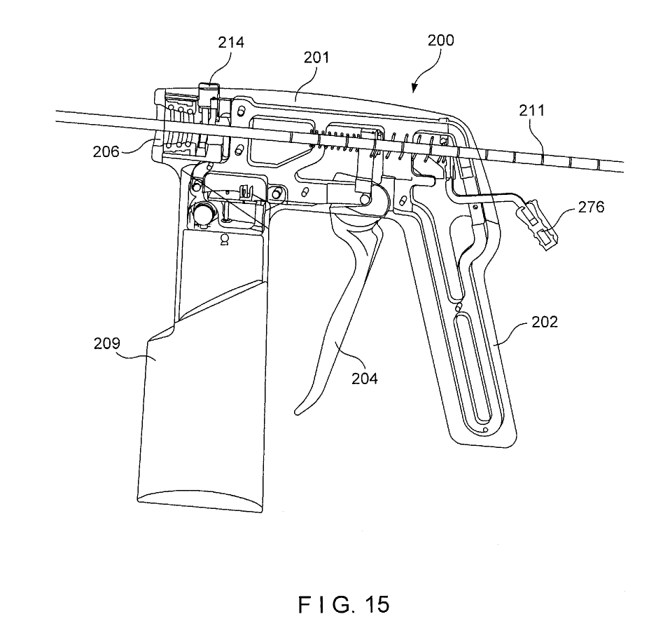

As shown in FIGS. 14-15, a delivery system 200 according to another exemplary embodiment of the present invention is substantially similar to the delivery system 100 described above and comprises a housing 201 surrounding the mechanisms of the delivery system. The system 200 comprises a handle 202 having an actuator 204 at a proximal end of the housing 201 and a coupling head 206 at a distal end of the housing 201. The handle 202 may, for example, be integrally formed with the housing 201. The coupling head 206 may also be integrally formed within the distal end of the housing 201 and is configured to be coupled to a cartridge 208 substantially similar to the cartridge 108 via which a bone strengthening material such as PCL is delivered to a bone. A toggle 214 of the coupling head 206, which facilitates connection to the cartridge 208, extends to an exterior of the housing 201 such that it is accessible to a user. A user presses the toggle 214 to release the cartridge 208. The delivery system 200 also similarly comprises a push rod 211 received within the housing 201 in alignment with the cartridge 208 such that moving the push rod distally via the actuator 204 moves the plunger assembly 164 and, consequently, the PCL rod 166 distally to force the melted PCL out of the cartridge 208 into the bone. The push rod 211 may be locked and unlocked relative to the housing 201 via a lever 276, substantially similar to the lever 176. The delivery system 200 may be connectable to a power source such as, for example, a rechargeable battery 209 which may, for example, be releasably coupled to the housing 201. In an exemplary embodiment, the battery 209 is coupled to the proximal end of the housing 201 so as not to interfere with the gripping of the handle 202 and actuation of the actuator 204. The battery 209 may be decoupled from the body 201 via a release button 213. It will be understood by those of skill in the art that the delivery system 200 may be utilized in a manner substantially similar to the delivery system 100, as described above.

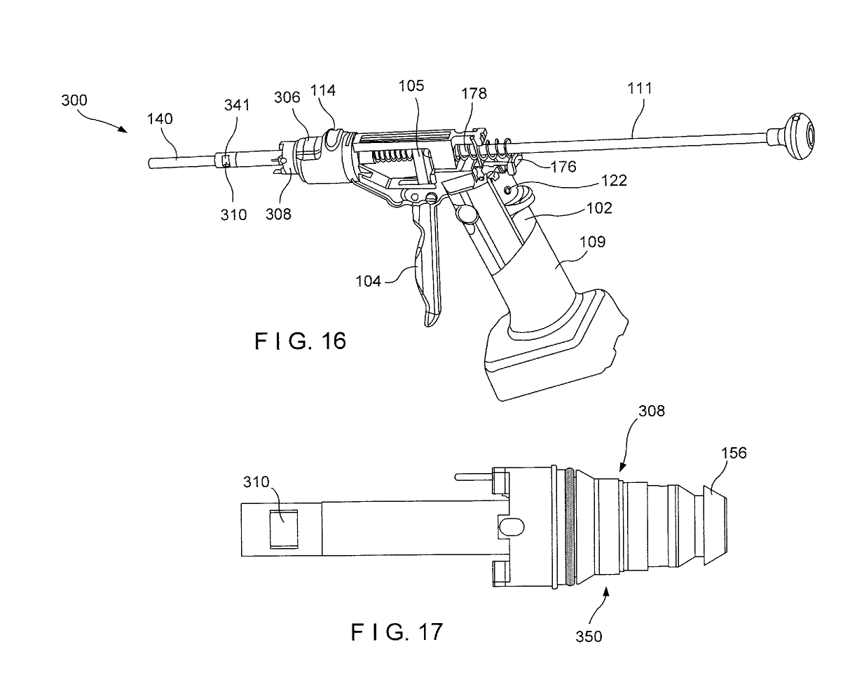

FIGS. 16-17 depict a system 300 according to yet another embodiment of the invention, wherein like elements have been referenced with like reference numerals. The system 300 is substantially similar to the system 100 but includes mechanisms which are not entirely encased within the body portion 101. The system 300 also comprises a cartridge 308 formed substantially similar to the cartridge 108 and engaging a coupling head 306. The cartridge 308 is configures to facilitate connection with the delivery tube 140. Specifically a portion of the shaft 136 of the delivery tube 140 may include a mating tab 342 configured to lockingly engage an opening 310 formed on the cartridge 308. Although the opening 310 and mating 342 are depicted with a substantially rectangular shape, any other shape may be used without deviating from the scope of the invention (e.g., circular, etc.). A distal portion of the neck 350 may be formed with a stepped shape selected to mate with a corresponding shape of the enlarged diameter portion 134 (not shown in FIGS. 16-17) attached the delivery tube 140. It is noted that the shape depicted herein is exemplary only and that the distal portion of the neck 350 and the proximal portion of the enlarged diameter portion 134 may assume any other shape without deviating the scope of the invention.

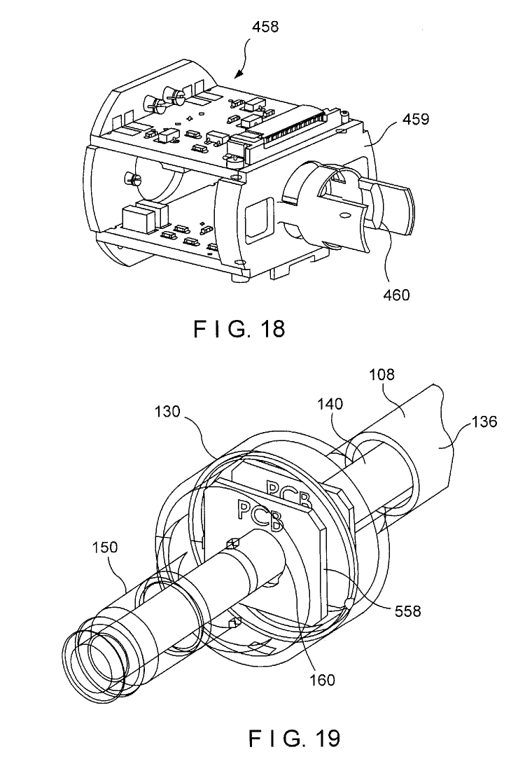

In another embodiment of the invention, as shown in FIG. 18, the PCB may be a multi-unit PCB 458 having a plurality of walls electrically connected to one another in an arrangement conforming to a shape and size of the enlarged diameter portion 134. The PCB 1458 may further comprise a support brace 459 having an opening 460 extending therethrough to receive the delivery tube 140. In yet another embodiment, as shown in FIG. 19, a PCB 558 may extend perpendicular to a longitudinal axis of the delivery tube 140 and have the opening 160 extending therethrough to receive the delivery tube 140.

It will be apparent to those skilled in the art that various modifications and variations may be made in the structure and the methodology of the present invention, without departing from the spirit or scope of the invention. Thus, it is intended that the present invention cover modifications and variations of the invention provided that they come within the scope of the appended claims and their equivalents.

What is claimed is:

1. A device for bone fixation, comprising:
a delivery gun having a handle;
an elongated cartridge coupled to the handle, the cartridge having a distal portion configured and dimensioned for insertion to a target area within a bone, the cartridge having a channel extending therethrough between a proximal end and an opening at the distal end and housing an injectable material therein, a first portion of the cartridge having a first heating element maintaining the injectable material within a first target temperature range and a second portion of the cartridge having a second heating element maintaining the injectable material within a second target temperature range.

2. The device of claim 1, wherein the second heating element is provided adjacent the distal end of the cartridge.

3. The device of claim 1, wherein the first target temperature range is between 80 degrees and 85 degrees C.

4. The device of claim 1, wherein the second target temperature range is between 60 degrees and 65 degrees C.

5. The device of claim 1, wherein the delivery gun includes an actuating mechanism moving the injectable material distally through the cartridge and out of the opening in the distal end of the cartridge.

6. The device of claim 5, wherein the actuating mechanism comprises a lever connected to a push rod slidably received within the cartridge.

7. The device of claim 6, wherein a proximal end of the injectable material is connected to a plunger, the push rod engaging the plunger to cause a distal movement of the injectable material.

8. The device of claim 1, wherein the delivery gun comprises a coupling head configured to removably couple the cartridge thereto.

9. The device of claim 8, wherein the coupling head comprises a switch movable between an open and a locking configuration to lockingly engage the cartridge.

10. The device of claim 1, wherein the cartridge comprises an indicator configured to indicate at least one of whether a temperature of injectable material in the reservoir is within the first target temperature range and whether a temperature of injectable material flowing through the cartridge is within the second target temperature range.

11. The device of claim 1, wherein the injectable material is one of polycaprolactone (PCL), ethylene vinyl acetate (EVA) and a thermoplastic.

12. A method for bone fixation, comprising:
inserting a cartridge of a bone fixation device to a target area in which a distal end thereof is adjacent to a target location within a bone, the bone fixation device comprising an injection gun having a handle, the cartridge being coupled to the handle and having a channel extending therethrough between a proximal end open and an opening at the distal end;
operating a first heating element within a first portion of the cartridge to maintain a temperature of an injectable material in the channel of the cartridge within a first target temperature range;
operating a distal portion heating arrangement in a distal portion of the cartridge to maintain a temperature of the injectable material flowing therethrough within a second target temperature range, the first and second target temperature ranges being selected to maintain the injectable material in a flowable state; and
moving the injectable material distally through the cartridge and out of the opening in the distal end of the cartridge into the target location within the bone.

13. The method according to claim 12, wherein the injectable material is one of polycaprolactone (“PCL”), ethylene vinyl acetate (EVA) and a thermoplastic.

14. The method according to claim 12, wherein the first temperature range is higher than the second temperature range and wherein a rate of injection of the injectable material is selected to permit the injectable material to cool from the first target temperature range to the second target temperature range before exiting the opening in the distal end of the cartridge.

15. The method according to claim 12, wherein the first heating element extends along a proximal portion of the cartridge which is coupled to the handle adjacent the reservoir.

16. The method according to claim 12, wherein the first target temperature ranges from between approximately 80° and 85° C.

17. The method according to claim 12, wherein the second target temperature ranges from between approximately 60° and 65° C.

18. The method according to claim 12, wherein the injectable material is moved out of the reservoir via an actuator which moves a plunger slidably received within the reservoir distally relative thereto.

19. The method according to claim 12, further comprising the step of rotating the delivery gun at any angle about a longitudinal axis of the cartridge without rotating the cartridge.

20. The method according to claim 12, further comprising the step of inserting the cartridge through an opening in an implanted bone fixation implant prior to injection of the injectable material.

21. A system for bone fixation, comprising:
a delivery gun having a handle;
an elongated cartridge coupled to the handle, the cartridge having a distal portion configured and dimensioned for insertion to a target area within a bone, the cartridge having a channel extending therethrough between a proximal end and an opening at the distal end;
an injectable material plunger having a plunger at a proximal end and an elongated rod of injectable material extending distally therefrom, the injectable material plunger configured for insertion through the channel of the cartridge, a first portion of the cartridge having a first heating element maintaining the injectable material within a first target temperature range and a second portion of the cartridge having a second heating element maintaining the injectable material within a second target temperature range; and
a push rod insertable through the delivery gun and into the channel of the cartridge to apply a distally directed force to the plunger to force the injectable material in a distal direction.

22. The system of claim 21, wherein the delivery gun includes an actuating mechanism moving the push rod.

23. The system of claim 21, wherein the delivery gun comprises a coupling head configured to removably couple the cartridge thereto.

24. The system of claim 22, wherein the coupling head comprises a switch movable between an open and a locking configuration to lockingly engage the cartridge.
